(19) United States
(12) Patent Application Publication
(10) Pub. No.: US 2022/0348543 A1
(43) Pub. Date: Nov. 3, 2022

(54) SALTS OF OMECAMTIV MECARBIL AND SOLID FORMS THEREOF

(71) Applicant: SYNTHON B.V., Nijmegen (NL)

(72) Inventor: Katerina JELINKOVA, Blansko (CZ)

(21) Appl. No.: 17/761,900

(22) PCT Filed: Sep. 18, 2020
(86) PCT No.: PCT/EP2020/076162

§ 371 Date: Mar. 18, 2022

(30) Foreign Application Priority Data
Sep. 19, 2019 (EP) 19198449.1

(51) Int. Cl. C07D 213/75

(52) U.S. Cl. CPC C07D 213/75; C07B 2200/13

(57) ABSTRACT

The presented invention relates to Omecamtiv mecarbil salts with methane sulfonic acid, benzene sulfonic acid and p-toluene sulfonic acid and to processes for preparation thereof.

### BACKGROUND OF THE PRESENT INVENTION

This invention relates to salts of Omecamtiv mecarbil, compound of formula (1), solid forms thereof and processes for preparation thereof;

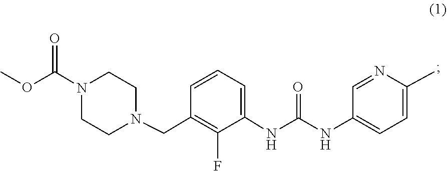

Omecamtiv mecarbil, 4-[2-Fluoro-3-[3-(6-methylpyridin-3-yl)ureido]benzyl]piperazine-1-carboxylic acid methyl ester, a small-molecule activator of cardiac myosin. Omecamtiv mecarbil is in phase III of clinical development for the oral treatment of chronic heart failure.

Omecamtiv mecarbil was disclosed in WO2006009726 application. WO2014152270 application discloses Omecamtiv mecarbil dihydrochloride salt, monohydrate thereof and solid forms thereof.

Solubility of API or its salt plays a major role for final dosage forms like parenteral or oral formulations. Solubility is one of the important parameters to achieve desired concentration of drug in systemic circulation for achieving required pharmacological response. Any drug to be absorbed must be present in the form of an aqueous solution at the site of absorption. For orally administered drugs solubility is the most important one rate limiting parameter to achieve their desired concentration in systemic circulation for pharmacological response. It is therefore advantageous to develop Omecamtiv mecarbil salts having improved solubility, purity or stability.

### BRIEF DESCRIPTION OF THE INVENTION

The presented invention relates to Omecamtiv mecarbil methane sulfonic acid salt, a solid form thereof and a process for preparation thereof.

The presented invention further relates to Omecamtiv mecarbil p-toluene sulfonic acid salt, a solid form thereof and a process for preparation thereof.

The presented invention also relates to Omecamtiv mecarbil benzenesulfonic acid salt, a solid form thereof and a process for preparation thereof.

The presented invention further relates to a pharmaceutical composition comprising Omecamtiv mecarbil salt of the presented invention.

The presented invention also relates to a use of the solid forms for purification of Omecamtiv mecarbil.

Omecamtiv mecarbil salt of the presented invention show improved solubility, crystallinity, purity and stability.

### BRIEF DESCRIPTION OF THE DRAWINGS

FIG. 1 depicts the X-Ray Powder Diffractogram (XRPD) of Omecamtiv mecarbil methane sulfonic acid salt, Form M, prepared according to Example 1.

FIG. 2 depicts the X-Ray Powder Diffractogram (XRPD) of Omecamtiv mecarbil p-toluene sulfonic acid salt, Form 1, prepared according to Example 3.

FIG. 3 depicts the X-Ray Powder Diffractogram (XRPD) of Omecamtiv mecarbil benzenesulfonic acid salt, Form B, prepared according to Example 2.

FIG. 4 depicts the DSC pattern of Omecamtiv mecarbil methane sulfonic acid salt, Form M, prepared according to Example 1.

FIG. 5 depicts the DSC pattern of Omecamtiv mecarbil p-toluene sulfonic acid salt, Form 1, prepared according to Example 3.

FIG. 6 depicts the DSC pattern of Omecamtiv mecarbil benzene sulfonic acid salt, Form B, prepared according to Example 2.

FIG. 7 depicts NMR spectrum of Omecamtiv mecarbil methane sulfonic acid salt, Form M, prepared according to Example 1.

FIG. 8 depicts NMR spectrum of Omecamtiv mecarbil p-toluene sulfonic acid salt, Form 1, prepared according to Example 3.

FIG. 9 depicts NMR spectrum of Omecamtiv mecarbil benzenesulfonic acid salt, Form B, prepared according to Example 2.

### DETAILED DESCRIPTION OF THE INVENTION

The presented invention relates Omecamtiv mecarbil methane sulfonic acid salt, a solid form thereof and a process for preparation thereof.

The solid form, Form M can be characterized by XRPD pattern having 2θ values 8.3°, 12.1° and 22.4° degrees 2 theta (±0.2 degrees 2 theta). The solid Form M can be also characterized by XRPD pattern having 2θ values 8.3°, 12.1°, 17.0°, 17.4° and 22.4° degrees 2 theta (±0.2 degrees 2 theta). The solid form can be further characterized by XRPD pattern described in the following table:

| Angle | Intensity |
|---|---|
| 2-Theta ° | % |
| 8.3 | 46.5 |
| 8.9 | 8.3 |
| 9.6 | 5.8 |
| 10.8 | 8.3 |
| 11.1 | 3.6 |
| 11.6 | 21.4 |
| 12.1 | 72.8 |
| 13.1 | 3.0 |
| 14.3 | 10.2 |
| 14.8 | 7.5 |
| 15.3 | 6.3 |
| 15.5 | 7.2 |
| 16.2 | 9.3 |
| 16.6 | 17.4 |
| 17.0 | 40.7 |
| 17.4 | 41.3 |
| 17.7 | 27.2 |
| 18.4 | 3.5 |
| 19.2 | 12.6 |
| 19.7 | 19.4 |
| 20.2 | 25.5 |
| 20.6 | 32.0 |
| 21.6 | 19.2 |
| 22.0 | 14.8 |
| 22.4 | 100.0 |
| 23.1 | 3.9 |
| 23.4 | 4.8 |
| 23.9 | 9.0 |
| 24.6 | 20.8 |
| 25.0 | 43.7 |
| 25.6 | 34.0 |
| 25.9 | 10.4 |
| 26.1 | 7.3 |
| 26.4 | 4.2 |
| 26.8 | 6.0 |
| 27.1 | 11.9 |
| 27.3 | 15.1 |
| 27.8 | 4.3 |
| 28.8 | 3.5 |
| 29.2 | 5.6 |
| 29.4 | 7.6 |
| 29.8 | 4.1 |
| 31.1 | 4.1 |
| 31.2 | 4.4 |
| 32.0 | 2.8 |
| 32.4 | 3.1 |
| 33.0 | 4.8 |
| 33.5 | 6.2 |
| 33.8 | 3.5 |
| 34.4 | 4.6 |

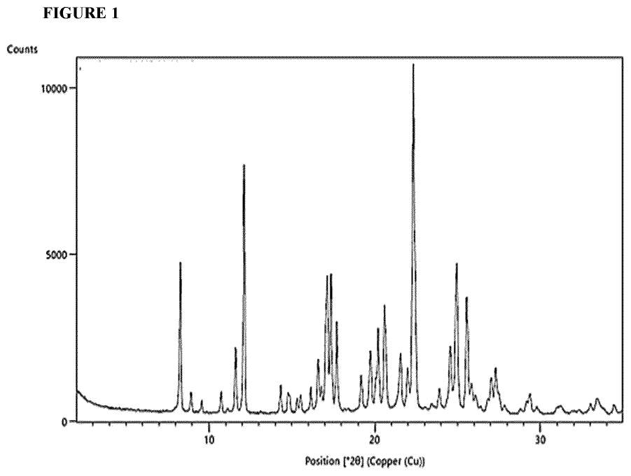

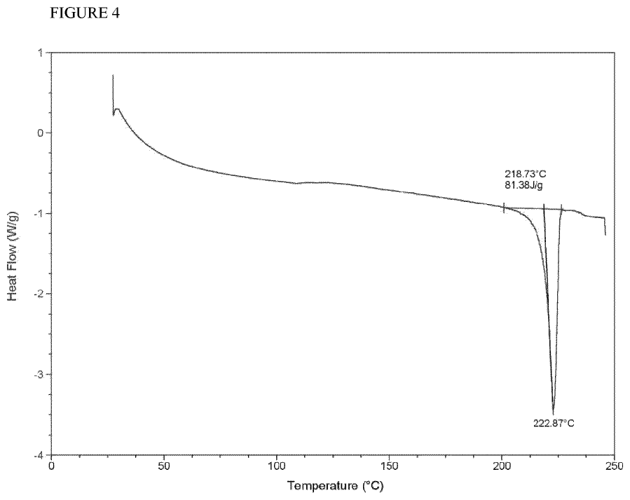

The solid form can be also characterized by XRPD pattern depicted in FIG. 1. The solid form can be further characterized by DSC pattern depicted in FIG. 4.

The solid form M can be prepared by a process comprising:
a. Dissolving of Omecamtiv mecarbil in 2-butanone;
b. Adding of methane sulfonic acid;
c. Isolating the solid form.

The concentration of Omecamtiv mecarbil in 2-butanone can be between 0.01 and 0.03 g/ml. Omecamtiv mecarbil can be dissolved at an elevated temperature for example between 50° C. and 90° C., preferably between 60° C. and 85° C. To the solution methane sulfonic acid is added. The molar ratio between Omecamtiv mecarbil and methanesulfonic acid can be between 1:1 and 1:1.3, preferably it is 1:1. The mixture is cooled to a temperature between −30° C. and 40° C., preferably between 0° C. and 30° C., more preferably between 15° C. and 25° C. and stirred for between 5 and 20 hours. The solid form can be isolated by any suitable technique, for example using filtration or centrifuge.

The preparation of Omecamtiv mecarbil methane sulfonic acid salt was tested also in following solvent: methanol, ethanol, isopropanol, 1-butanol, acetone and tetrahydrofurane. Solid obtainable from these solvents was not stable and it melted at a temperature 25° C. The purity of obtained solid Form M is significantly improved comparing to purity of starting Omecamtiv mecarbil. The solid Form M can be therefore used for purification of Omecamtiv mecarbil. The salt can be transformed into Omecamtiv mecarbil by contacting with a base (for example sodium hydrogen carbonate or potassium hydrogen carbonate or sodium carbonate or potassium carbonate or sodium hydroxide or potassium hydroxide) in a suitable solvent (for example an alcohol or acetone).

The presented invention further relates to Omecamtiv mecarbil p-toluene sulfonic acid salt, a solid form thereof and a process for preparation thereof. The solid form, Form 1, can be characterized by XRPD pattern having 2θ values 5.9°, 15.7° and 16.6° degrees 2 theta (±0.2 degrees 2 theta). The solid Form 1 can be also characterized by XRPD pattern having 20 values 5.9°, 15.7°, 16.1°, 16.6° and 17.9° degrees 2 theta (±0.2 degrees 2 theta). The solid form can be further characterized by XRPD pattern described in the following table:

| Angle | Intensity |
|---|---|
| 2-Theta ° | % |
| 5.0 | 7.0 |
| 5.9 | 100.0 |
| 7.0 | 2.3 |
| 7.9 | 3.6 |
| 9.5 | 2.5 |
| 10.1 | 3.5 |
| 10.5 | 5.4 |
| 11.8 | 2.7 |
| 12.2 | 3.6 |
| 12.7 | 4.5 |
| 13.2 | 4.5 |
| 13.9 | 6.6 |
| 14.1 | 5.7 |
| 14.8 | 6.1 |
| 15.4 | 8.1 |
| 15.7 | 14.8 |
| 16.1 | 11.1 |
| 16.6 | 14.7 |
| 17.2 | 12.8 |
| 17.9 | 14.3 |
| 18.3 | 8.6 |
| 19.1 | 6.8 |
| 19.7 | 6.0 |
| 20.5 | 14.3 |
| 21.1 | 12.3 |
| 21.5 | 7.5 |
| 22.2 | 6.0 |
| 23.0 | 5.2 |
| 23.7 | 6.9 |
| 24.2 | 8.3 |
| 25.0 | 11.2 |
| 25.6 | 12.6 |
| 26.6 | 5.5 |
| 27.4 | 8.0 |
| 27.8 | 8.3 |
| 28.5 | 4.8 |
| 29.5 | 4.0 |
| 30.2 | 3.7 |
| 31.6 | 2.7 |

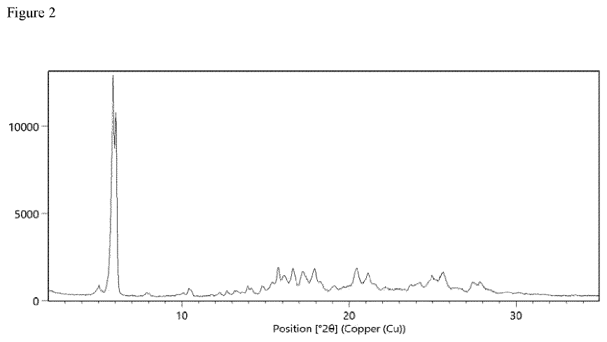

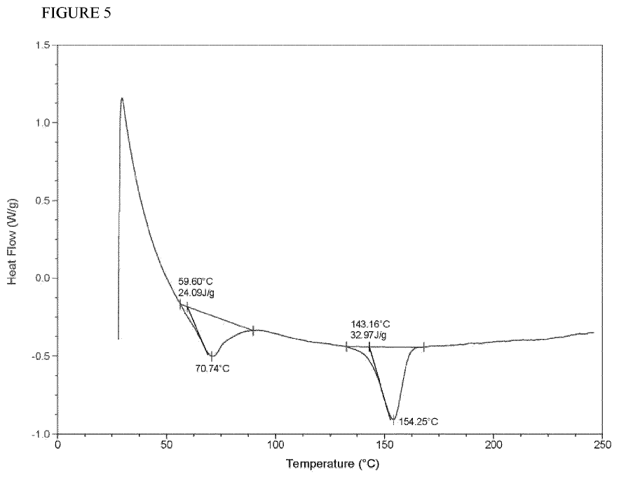

The solid Form 1 can be further characterized by XRPD pattern depicted in FIG. 2 and DSC pattern depicted in FIG. 5. The solid form of Omecamtiv mecarbil p-toluene sulfonic acid salt can be prepared by a process comprising:
a. Dissolving of Omecamtiv mecarbil in a solvent selected from an alcohol or acetone or 2-butanone or tetrahydrofurane;
b. Adding of p-toluene sulfonic acid;
c. Isolating the solid form.

The alcohol is step a. can be selected from methanol or ethanol or propanol or isopropanol or 1-butanol or 2-butanol. The concentration of Omecamtiv mecarbil in the solvent can be:
1. In case the solvent is an alcohol between 0.04 g/ml and 0.11 g/ml, preferably it is between 0.07 g/ml and 0.11 g/ml;
2. In case the solvent is acetone between 0.007 g/ml and 0.02 g/ml, preferably between 0.009 g/ml and 0.012 g/ml;
3. In case the solvent is 2-butanone between 0.008 g/ml and 0.05 g/ml, preferably between 0.03 g/ml and 0.05 g/ml;
4. In case the solvent is tetrahydrofurane between 0.008 g/ml and 0.04 g/ml, preferably between 0.02 g/ml and 0.04 g/ml.

Omecamtiv mecarbil can be dissolved at an elevated temperature for example between 50° C. and 120° C., preferably between 60° C. and 100° C. To the solution p-toluene sulfonic acid is added. P-toluene sulfonic acid can be added as solid or in form of a solution in a solvent. In case p-toluene sulfonic acid is used as a solution the concentration of the solution can be between 0.07 and 0.2 g/ml. The molar ration between Omecamtiv mecarbil and p-toluene sulfonic acid can be between 1:1 and 1:1.3, preferably it is 1:1.

The mixture is cooled to a temperature between −30° C. and 40° C., preferably between 0° C. and 30° C., more preferably between 15° C. and 25° C. and stirred for between 5 and 20 hours. The solid form can be isolated by any suitable technique, for example using filtration or centrifuge.

The purity of obtained solid Form 1 is significantly improved comparing to purity of starting Omecamtiv mecarbil. The solid Form 1 can be therefore used for purification of Omecamtiv mecarbil. The salt can be transformed into Omecamtiv mecarbil by contacting with a base (for example sodium hydrogen carbonate or potassium hydrogen carbonate or sodium carbonate or potassium carbonate or sodium hydroxide or potassium hydroxide) in a suitable solvent.

The presented invention further relates to Omecamtiv mecarbil benzene sulfonic acid salt, a solid form thereof and a process for preparation thereof.

The solid form, Form B, can be characterized by XRPD pattern having 2θ values 5.4°, 16.3° and 17.8° 2 theta (±0.2 degrees 2 theta). The solid Form B can be also characterized by XRPD pattern having 2θ values 5.4°, 16.3°, 17.0° and 17.8° degrees 2 theta (±0.2 degrees 2 theta). The solid form can be further characterized by XRPD pattern described in the following table:

| Angle | Intensity |
|---|---|
| 2-Theta ° | % |
| 5.4 | 100.0 |
| 5.9 | 29.6 |
| 7.5 | 12.0 |
| 7.8 | 23.1 |
| 8.4 | 27.2 |
| 10.2 | 11.0 |
| 10.8 | 14.0 |
| 11.9 | 10.4 |
| 12.3 | 16.3 |
| 13.3 | 10.7 |
| 13.9 | 12.7 |
| 14.3 | 16.3 |
| 14.8 | 17.4 |
| 15.6 | 17.8 |
| 16.3 | 36.5 |
| 17.0 | 25.7 |
| 17.8 | 43.5 |
| 18.5 | 26.0 |
| 19.2 | 21.4 |
| 20.1 | 24.2 |
| 20.6 | 27.4 |
| 21.3 | 34.6 |
| 21.9 | 30.2 |
| 22.6 | 32.8 |
| 23.5 | 17.8 |
| 23.8 | 18.1 |
| 24.4 | 21.4 |
| 25.0 | 14.7 |
| 25.7 | 13.8 |
| 26.6 | 15.5 |
| 27.2 | 27.5 |
| 28.0 | 13.8 |
| 28.7 | 11.4 |
| 29.2 | 10.4 |
| 30.2 | 9.2 |
| 31.1 | 9.7 |
| 32.1 | 8.3 |
| 33.5 | 6.3 |
| 33.6 | 7.2 |

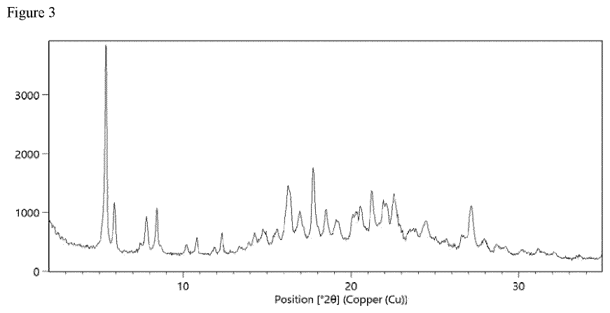

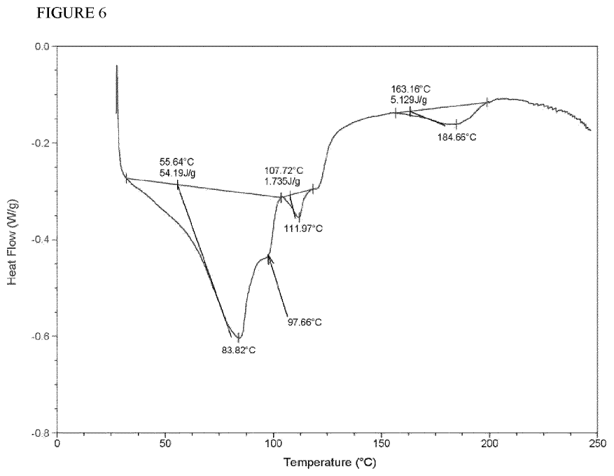

The solid Form B can be further characterized by XRPD pattern depicted in FIG. 3 and DSC pattern depicted in FIG. 6. The solid form of Omecamtiv mecarbil benzene sulfonic acid salt can be prepared by a process comprising:
a. Dissolving of Omecamtiv mecarbil in acetone;
b. Adding of benzene sulfonic acid;
c. Isolating the solid form.

The concentration of Omecamtiv mecarbil in acetone can be between 0.007 g/ml and 0.02 g/ml, preferably between 0.009 g/ml and 0.012 g/ml. Omecamtiv mecarbil can be dissolved at an elevated temperature for example between 40° C. and 56° C. To the solution benzene sulfonic acid is added. Benzene sulfonic acid can be added as solid or in form of a solution in acetone. In case benzene sulfonic acid is used as a solution the concentration of the solution can be between 0.07 and 0.22 g/ml. The molar ration between Omecamtiv mecarbil and benzene sulfonic acid can be between 1:1 and 1:2.1.

The mixture is cooled to a temperature between −30° C. and 0° C., preferably between −20° C. and −10° C., and stirred for between 1 and 20 days. The solid form can be isolated by any suitable technique, for example using filtration or centrifuge.

The preparation of Omecamtiv mecarbil benzene sulfonic acid salt was tested also in following solvent: methanol, ethanol, isopropanol, 1-butanol, 2-butanone and tetrahydrofurane. No solid form appeared.

The purity of obtained solid Form B is significantly improved comparing to purity of starting Omecamtiv mecarbil. The solid Form B can be therefore used for purification of Omecamtiv mecarbil. The salt can be transformed into Omecamtiv mecarbil by contacting with a base (for example sodium hydrogen carbonate or potassium hydrogen carbonate or sodium carbonate or potassium carbonate or sodium hydroxide or potassium hydroxide) in a suitable solvent (for example an alcohol or tetrahydrofurane).

A preparation of salts of Omecamtiv mecarbil with acetic acid or benzoic acid was tested using above disclosed process. Only free form of Omecamtiv mecarbil was obtained.

The salts of presented invention can be used in a pharmaceutical composition for the treatment of conditions treatable by Omecamtiv mecarbil. The salts of presented invention can be also used for purification of Omecamtiv mecarbil.

The invention will be further described with reference to the following examples.

### EXAMPLES

Nuclear magnetic resonance spectroscopy (NMR) was performed using Avance III 400 MHz NMR spectrometer.

DCS patterns were obtained using the following conditions: 10° C./min->250° C.

XRPD spectrum was obtained using the following measurement conditions:

Panalytical Empyrean diffractometer with Θ/2Θ geometry (transmission mode), equipped with a PixCell 3D detector;

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406 Å (Kα1), primary monochromator used | |
| Divergence slit: | 1/2° | |
| Antiscatter slit: | 1/2° | |
| Soller slit: | 0.02 | rad |
| Detector slit: | 7.5 | mm |
| Rotation speed: | 30 | rpm |

### Example 1: Omecamtiv Mecarbil Salt with Methanesulfonic Acid, Form M

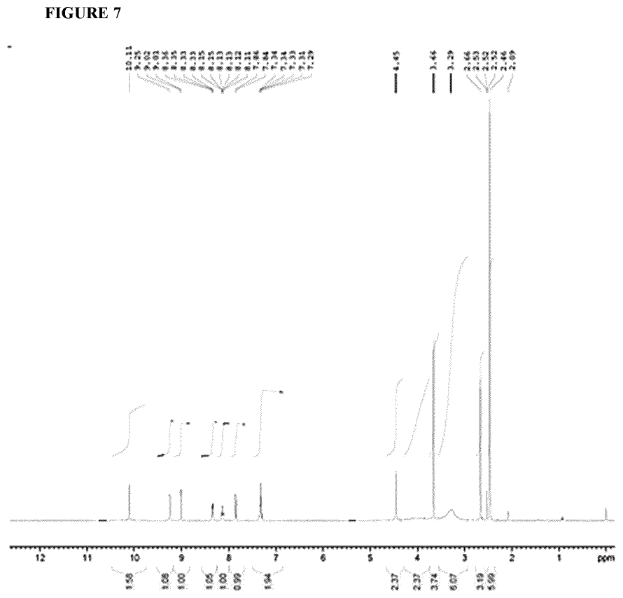

250 mg of Omecamtiv mecarbil was dissolved in a solvent (in Table below) at a temperature (in Table below). 40 μl methane sulfonic acid (1 eq. w.r.t. Omecamtiv mecarbil) was added. The mixture was cooled to 25° C. and stirred for 16 hours. If no solid appeared, mixture was then stirred at −15° C. Solid mass was filtered off and dried. The ratio between Omecamtiv mecarbil and the salt was determined by NMR as 1:2 (i.e. dimethane sulfonate salt).

| | Volume of | | | | |
|---|---|---|---|---|---|
| Solvent | solvent | | Temperature | | Results |
| 2-Butanone | 10 | ml | 80° | C. | Crystals appeared at 25° C. |
| | | | | | after 16 hours, Crystalline |
| | | | | | form M, stable at 25° C., |
| | | | | | XRPD depicted in FIG. 1, |
| | | | | | DSC depicted in FIG. 4, |
| | | | | | NMR depicted in FIG. 7 |
| Methanol | 3.5 | ml | 65° | C. | Crystals appeared after |
| | | | | | 7 days at −15° C., |
| | | | | | but it melted at 25° C. |
| Ethanol | 2.5 | ml | 78° | C. | Crystals appeared after |
| | | | | | 2 days at −15° C., |
| | | | | | but it melted at 25° C. |
| isopropanol | 5.0 | ml | 82° | C. | Crystals appeared after |
| | | | | | 2 days at −15° C., |
| | | | | | but it melted at 25° C. |
| 1-Butanol | 3.0 | ml | 100° | C. | Crystals appeared after |
| | | | | | 2 days at −15° C., |
| | | | | | but it melted at 25° C. |
| Acetone | 20 | ml | 56° | C. | Crystals appeared after |
| | | | | | 7 days at −15° C., |
| | | | | | it melted at 25° C. |
| Tetrahydro- | 6.5 | ml | 66° | C. | Omecamtiv mecarbil free |
| furane | | | | | base appeared after 1 |
| | | | | | month at −15° C. |

### Example 2: Omecamtiv Mecarbil Salt with Benzenesulfonic Acid, Form B

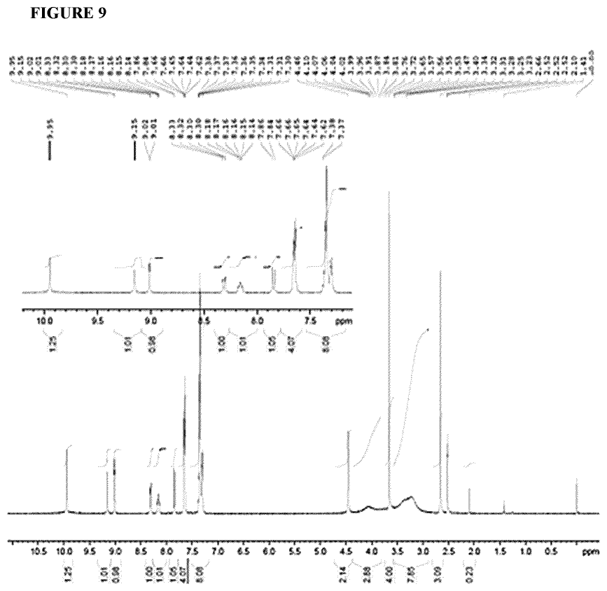

250 mg of Omecamtiv mecarbil was dissolved in a solvent (in Table below) at a temperature (in Table below). 99 mg of benzene sulfonic acid (1 eq. w.r.t. Omecamtiv mecarbil) in 1 ml of the solvent was added. The mixture was cooled to 25° C. and stirred for 16 hours. If no solid appeared, mixture was stirred at −15° C. Solid mass was filtered off and dried. The procedure was repeated with 2 eq. of benzenesulfonic acid (w.r.t. Omecamtiv mecarbil) with the same results (only in acetone Form B was prepared). The ratio between Omecamtiv mecarbil and the salt was determined by NMR as 1:2 (i.e. dibenzene sulfonate salt).

| | Volume of | | | | |
|---|---|---|---|---|---|
| Solvent | solvent | | Temperature | | Results |
| Acetone | 20 | ml | 56° | C. | Crystals were observed after |
| | | | | | 10 days at −15° C., Form |
| | | | | | B, XRPD depicted in FIG. 3, |
| | | | | | DSC depicted in FIG. 6, NMR |
| | | | | | depicted in FIG. 9 |
| Methanol | 3.5 | ml | 65° | C. | No crystals |
| Ethanol | 2.5 | ml | 78° | C. | No crystals |
| Isopropanol | 5.0 | ml | 82° | C. | Oil |
| 1-Butanol | 3.0 | ml | 100° | C. | Oil |
| 2-Butanone | 10 | ml | 80° | C. | Oil |
| Tetrahydro- | 6.5 | ml | 66° | C. | Oil |
| furane | | | | | |

### Example 3: Omecamtiv Mecarbil Salt with p-Toluene Sulfonic Acid, Form 1

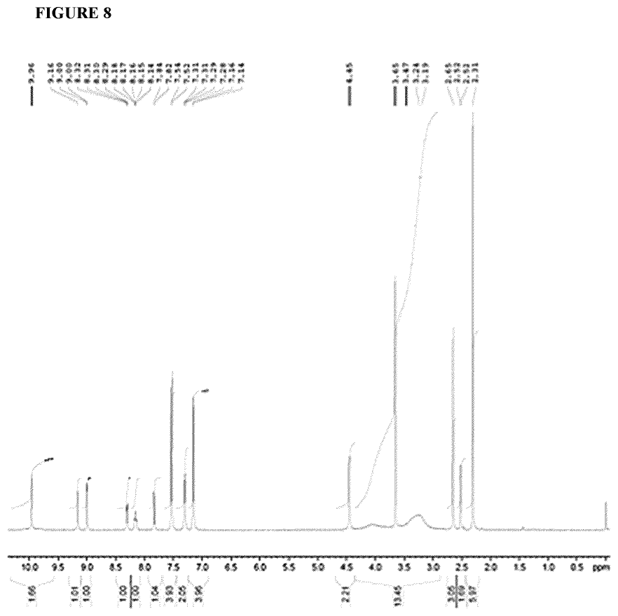

250 mg of Omecamtiv mecarbil was dissolved in a solvent (in Table below) at a temperature (in Table below). 214 mg of p-toluene sulfonic acid (2 eq. w.r.t. Omecamtiv mecarbil) in 1 ml of the solvent was added. The mixture was cooled to 25° C. and stirred for 16 hours. If no solid appeared, mixture was stirred at −15° C. Solid mass was filtered off and dried. XRPD of prepared Form 1 is depicted in FIG. 2, DSC is depicted in FIG. 5, NMR is depicted in FIG. 8. The ratio between Omecamtiv mecarbil and the salt was determined by NMR as 1:2 (i.e. di p-toluene sulfonate salt).

| | Volume of | | | | |
|---|---|---|---|---|---|
| Solvent | solvent | | Temperature | | Results |
| Ethanol | 2.5 | ml | 78° | C. | Crystalline Form 1 at 25° C. |
| Isopropanol | 5.0 | ml | 82° | C. | Crystalline Form 1 at 25° C. |
| 1-Butanol | 3.0 | ml | 100° | C. | Crystalline Form 1 at 25° C. |
| Acetone | 20 | ml | 56° | C. | Crystalline Form 1 at 25° C. |
| 2-Butanone | 10 | ml | 80° | C. | Crystalline Form 1 at 25° C. |
| Tetrahydro- | 6.5 | ml | 66° | C. | Crystalline Form 1 at 25° C. |
| furane | | | | | |
| Methanol | 3.5 | ml | 65° | C. | No crystals |

### Example 4: Solubility of Omecamtiv Mecarbil Salts

The solubility of prepared salts was tested in water solution at different pH (1.2, 4.5 and 6.8) and compared to Omecamtiv mecarbil dihydrochloride monohydrate salt prepared according to a procedure disclosed in WO2006009726 application. The results are summarized in the following table.

It can be concluded that solubility of all prepared salt is better that the solubility of dihydrochloride monohydrate salt.

Omecamtiv Mecarbil salts - Category of solubility (Descriptive Term)¹

| | Solvent (aqueous buffer) | | |
|---|---|---|---|
| | pH 1.2 | pH 4.5 | pH 6.8 |
| Salt with HCl | Freely soluble | Freely soluble | Freely soluble |
| | (100-1000 mg/mL) | (100-1000 mg/mL) | (100-1000 mg/mL) |
| Salt with benzene | Very soluble | Very soluble | Very soluble |
| sulfonic acid, Form B | (≥1000 mg/mL) | (≥1000 mg/mL) | (≥1000 mg/mL) |
| Salt with methane | Very soluble | Very soluble | Very soluble |
| sulfonic acid, Form M | (≥1000 mg/mL) | (≥1000 mg/mL) | (≥1000 mg/mL) |
| Salt with p-toluene | Very soluble | Very soluble | Very soluble |
| sulfonic acid, Form 1 | (≥1000 mg/mL) | (≥1000 mg/mL) | (≥1000 mg/mL) |

¹According to the USP, section Reference Tables - Description and Solubility, corresponds with the Ph. Eur., Chapter 1. General Notices, section 1.4 Monographs - Solubility

### Example 5: Purification of Omecamtiv Mecarbil Using Omecamtiv Mecarbil Salt with Methanesulfonic Acid (Form M)

1 g of Omecamtiv mecarbil was dissolved in 40 ml of 2-Butanone at 80° C. 160 μl of methane sulfonic acid (1 eq. w.r.t. Omecamtiv mecarbil) was added. The mixture was cooled to 25° C. and stirred for 16 hours. The mixture was then stirred at −15° C. for 3 hours. Solid mass was filtered off and dried. The purity (HPLC) of obtained methane sulfonate salt of Omecamtiv mecarbil was significantly higher than purity of starting Omecamtiv mecarbil.

### Example 6: Purification of Omecamtiv Mecarbil Using Omecamtiv Mecarbil Salt with Benzenesulfonic Acid (Form B)

1 g of Omecamtiv mecarbil was dissolved in 80 ml of acetone at 56° C. 396 mg of benzene sulfonic acid (1 eq. w.r.t. Omecamtiv mecarbil) in 4 ml of acetone was added. The mixture was cooled to 25° C. and stirred for 16 hours. The mixture was then stirred at −15° C. for 3 hours. Solid mass was filtered off and dried. The purity (HPLC) of obtained benzene sulfonate salt of Omecamtiv mecarbil was significantly higher than purity of starting Omecamtiv mecarbil.

### Example 7: Purification of Omecamtiv Mecarbil Using Omecamtiv Mecarbil Salt with p-Toluene Sulfonic Acid (Form 1)

1 g of Omecamtiv mecarbil was dissolved in 10 ml of Ethanol at 78° C. 856 mg of p-toluene sulfonic acid (2 eq. w.r.t. Omecamtiv mecarbil) in 4 ml of Ethanol was added. The mixture was cooled to 25° C. and stirred for 16 hours. The mixture was stirred at −15° C. for 3 hours. The purity (HPLC) of obtained p-toluene sulfonate salt of Omecamtiv mecarbil was significantly higher than purity of starting Omecamtiv mecarbil.

1. Omecamtiv mecarbil p-toluene sulfonic acid salt.

2. A solid form of Omecamtiv mecarbil p-toluene sulfonic acid salt.

3. The solid form according to claim 2, wherein said solid form is crystalline Form 1, characterized by XRPD pattern having 2θ values 5.9°, 15.7° and 16.6° degrees 2 theta (±0.2 degrees 2 theta).

4. The solid form according to claim 3, characterized by XRPD pattern having 2θ values 5.9°, 15.7°, 16.1°, 16.6° and 17.9° degrees 2 theta (±0.2 degrees 2 theta).

5. A process for preparation of the solid form according to claim 3, comprising:
a) dissolving of Omecamtiv mecarbil in an alcohol or acetone or 2-butanone or tetrahydrofurane;
b) adding of p-toluene sulfonic acid; and
c) isolating the solid form.

6. The process according to claim 5, wherein the alcohol is selected from methanol or ethanol or propanol or isopropanol or 1-butanol or 2-butanol.

7. Omecamtiv mecarbil benzene sulfonic acid salt.

8. A solid form of Omecamtiv benzene sulfonic acid salt.

9. The solid form according to claim 8, wherein said solid form is crystalline Form B, characterized by XRPD pattern having 2θ values 5.4°, 16.3°, 17.0° and 17.8° degrees 2 theta (±0.2 degrees 2 theta).

10. The solid form according to claim 9, characterized by XRPD pattern having 2θ values 5.4°, 5.9°, 16.3°, 17.0°, 17.8° and 18.5° degrees 2 theta (±0.2 degrees 2 theta).

11. A process for preparation of the solid form according to claim 9, comprising:
a) dissolving of Omecamtiv mecarbil in acetone;
b) adding of benzene sulfonic acid; and
c) isolating the solid form.

12. A solid form of Omecamtiv mecarbil methane sulfonic acid salt, wherein said solid Omecamtiv mecarbil methane sulfonic acid salt is crystalline form M, characterized by XRPD pattern having 2θ values 8.3°, 12.1° and 22.4° degrees 2 theta (±0.2 degrees 2 theta).

13. The solid form according to claim 12, characterized by XRPD pattern having 2θ values 8.3°, 12.1°, 17.0°, 17.4° and 22.4° degrees 2 theta (±0.2 degrees 2 theta).

14. A process for preparation of the solid form according to claim 12, comprising:
a) dissolving of Omecamtiv mecarbil in 2-butanone;
b) adding of methane sulfonic acid; and
c) isolating the solid form.

15. (canceled)
